(19) United States
(12) Patent Application Publication
(10) Pub. No.: US 2006/0165742 A1
(43) Pub. Date: Jul. 27, 2006

(54) Powdery active ingredient formulations

(76) Inventors: Karl Reizlein, Koln (DE); Bernd Klinksiek, Bergisch Gladbach (DE); Jurgen Hinderer, Oakdale, PA (US); Peter Baur, Eppstein (DE); Anne Suty-Heinze, Langenfeld (DE); Astrid Mauler-Machnik, Leichlingen (DE); Heike Bunjes, Jena (DE); Kirsten Westesen, Niederrossla (DE); Jonas Hinnen, Marburg (DE); Albert Hinnen, Marburg (DE)

Correspondence Address: BAYER CROPSCIENCE LP;Patent Department, 100 BAYER ROAD, PITTSBURGH, PA, 15205-9741, US

(21) Appl. No.: 10/492,674

(22) PCT Filed: Oct. 17, 2002
(86) PCT No.: PCT/EP02/11613

§ 371 Date: Nov. 28, 2005

(30) Foreign Application Priority Data
Oct. 18, 2001 (DE) 101 51 392.5

(51) Int. Cl. A01N 25/00; A61K 9/14

(52) U.S. Cl. 424/405; 424/489

(57) ABSTRACT

Novel pulverulent active substance formulations composed of at least one active substance which is solid at room temperature, at least one dispersant, polyvinyl alcohol and if desired, additives, the individual active substance particles being enveloped by a coat of polyvinyl alcohol, being present in the amorphous state, and having diameter in the nanometer range, a process for producing the novel formulations and their use for the application of the active substance they comprise and also a device for carrying out the production process.

The present invention relates to new pulverulent active substance formulations, to a process for preparing them and to their use for applying the biologically active substances they include.

Numerous active substance formulations have already been disclosed in which the active components are present in a very finely divided state.

For instance, WO 94-20 072 and U.S. Pat. No. 5,785,976 describe suspensions of solid active substances that are of low solubility in water. These formulations can be prepared by melting solid active substance, heating the dispersion medium to approximately the temperature of the active substance melt, adding a water-soluble stabilizer to the dispersion medium and optionally a lipid-soluble stabilizer to the active substance melt, then mixing and homogenizing melt and dispersion and cooling the resultant dispersion. The effectiveness of these formulations in the freshly prepared state is good. A disadvantage, however, is that the bioavailability of the active components leaves something to be desired.

WO 95-05 164, moreover, discloses formulations of sparingly water-soluble substances in the form of supercooled melts in which the substances are liquid, are at least partly in amorphous state and have diameters in the nanometer range. These formulations are, however, of limited stability owing to the partially crystalline state, since recrystallization may occur.

Furthermore, WO 98-16 105 discloses solid plant treatment compositions in which the active substances are very finely divided, are essentially in amorphous form and are surrounded by a polymeric coat. These formulations are obtained by mixing a liquid formulation of the active substance with a liquid formulation of the coating material and removing the solvent from the resultant mixture. The stability and efficacy of the formulations obtained in this case, however; are not always sufficient. Moreover, the procedure is very laborious, since not only solvent but considerable amounts of water too have to be removed.

Novel pulverulent active substance formulations have now been found which are composed of
at least one active substance which is solid at room temperature,
at least one dispersant,
polyvinyl alcohol, and
optionally additives,
   the individual active substance particles being coated with a coat of polyvinyl alcohol, being present in an amorphous state and having a diameter in the nanometer region.

It has further been found that the pulverulent active substance formulations of the invention can be produced by
a) suspending at least one active substance which is solid at room temperature, at least one dispersant, and optionally additives in water, at room temperature,
b) heating the resultant suspension until the solid components it comprises melt,
c) first homogenizing the dispersion obtained and then rapidly cooling it to a temperature below the solidification point of the dispersed components,
d) thereafter adding an aqueous solution of polyvinyl alcohol, alone or in a mixture with other coating materials and also, optionally, additives and immediately thereafter subjecting the resultant dispersion to spray drying.

Finally it has been found that the pulverulent active substance formulations of the invention are especially suitable for the application of the active substances they compnse.

It is considered extremely surprising that the pulverulent active substance formulations of the invention are substantially more stable than the existing preparations constitutionally closest to them, which are obtainable by melt dispersing, but in which the individual particles are not encapsulated. Another reason for the unexpectedness of the stability of the formulations of the invention is that it is assumed that when the powders are stirred together with water the polyvinyl alcohol coat dissolves, and it was therefore feared that the amorphous active substance, which would then be unprotected, would recrystallize. Contrary to expectations, however, this effect does not occur.

The pulverulent active substance formulations of the invention are also notable for a number of advantages. For instance, the active substance content is very high as compared with corresponding existing formulations. This means that just a small amount of formulation is sufficient to apply the desired quantity of active component. Another advantage is that the pulverulent active substance formulations of the invention can be redispersed readily prior to use and that the bioavailability of the active components remains at the high level achieved following production. It is favourable, finally, that the thermal load on the active substances during the production of the formulations is comparatively low. Moreover, the preparation of the powder formulations of the invention requires neither additional carrier materials nor organic solvents. Furthermore, the process of the invention can also be used without problems to process active substances whose melting point is well above 100° C.

Suitable active substances present in the pulverulent formulations of the invention are at room temperature solid active pharmaceutical substances, active agrochemical substances and aromas.

Examples that may be mentioned of active pharmaceutical substances are ibuprofen, clotrimazole, fluconazole, indoxacarb and ciprofloxazin.

By agrochemical substances in the present context are meant all substances customary for plant treatment whose melting point is above 20° C. With preference mention may be made of fungicides, bactericides, insecticides, acaricides, nematicides, molluscicides, herbicides and plant growth regulators.

Examples of Fungicides Which May be Mentioned are:

2-anilino-4-methyl-6-cyclopropylpyrimidine; 2′,6′-dibromo-2-methyl-4′-trifluoromethoxy-4′-trifluoromethyl-1,3-thiazole-5-carboxanilide; 2,6-dichloro-N-(4-trifluoromethylbenzyl)benzamide; (E)-2-methoximino-N-methyl-2-(2-phenoxyphenyl)acetamide; 8-hydroxyquinoline sulfate; methyl (E)-2-{2-[6-(2-cyanophenoxy)pyrimidine-4-yloxy]phenyl}-3-methoxyacrylate; methyl (E)-methoximino[alpha-(o-tolyloxy)-o-tolyl]acetate; 2-phenylphenol (OPP), ampropylfos, anilazin, azaconazole, benalaxyl, benodanil, benomyl, binapacryl, biphenyl, bitertanol, blasticidin-S, bromuconazole, bupirimate, buthiobate, captafol, captan, carbendazim, carboxin, chinomethionat (quinomethionate), chloroneb, chloropicrin, chlorothalonil, chlozolinate, cufraneb, cymoxanil, cyproconazole, cyprofuram, carpropamid, dichlorophen, diclobutrazole, dichlofluanid, diclomezin, dicloran, diethofencarb, difenoconazole, dimethirimol, dimethomorph, diniconazole, diphenylamine, dipyrithion, ditalimfos, dithianon, dodine, drazoxolon, epoxyconazole, ethirimol, etridiazole, fenarimol, fenbuconazole, fenfuram, fenitropan, fenpiclonil, fentin acetate, fentin hydroxide, ferbam, ferimzone, fluazinam, fludioxonil, fluoromide, fluquinconazole, flusilazole, flusulfamide, flutolanil, flutriafol, folpet, fosetyl-aluminium, fthalide, fuberidazole, furalaxyl, furmecyclox, fenhexamid, guazatine, hexachlorobenzene, hexaconazole, hymexazol, imazalil, imibenconazole, iminoctadin, iprodione, isoprothiolane, iprovalicarb, kasugamycin, mancopper, mancozeb, maneb, mepanipyrim, mepronil, metalaxyl, metconazole, methasulfocarb, methfuroxam, metiram, metsulfovax, myclobutanil, nickel dimethyldithiocarbamate, nitrothal-isopropyl, nuarimol, ofurace, oxadixyl, oxamocarb, oxycarboxin, penconazole, pencycuron, phosdiphen, pimaricin, polyoxin, probenazole, prochloraz, procymidone, propamocarb, propineb, pyrazophos, pyrimethanil, pyroquilon, quintozene (PCNB), quinoxyfen, tebuconazole, tecloftalam, tecnazen, thiabendazole, thicyofen, thiophanate-methyl, thiram, tolclophos-methyl, tolylfluanid, triadimefon, triadimenol, triazoxide, trichlamid, tricyclazole, triflumizole, triforine, triticonazole, trifloxystrobin validamycin A, vinclozolin, zineb, ziram, 2-[2-(1-chloro-cyclopropyl)-3-(2-chlorophenyl)-2-hydroxypropyl]-2,4-dihydro-[1,2,4]-triazole-3-thione

3-(1-[2-(4-[2-chlorophenoxy)-5-fluoropyrimid-o-yloxy)-phenyl]-1-(methoximino)-methyl)-5,6-dihydro-1,4,2,-dioxazine and

2-(2-[6-(3-chloro-2-methylphenoxy)-5-fluoropyrimid-4-yloxy]-phenyl)-2-methoximino-N-methyl-acetamide.

Examples of Bactericides Which May be Mentioned are:

bronopol, dichlorophen, nitrapyrin, octhilinone, furancarboxylic acid, oxytetracyclin, probenazole, tecloftalam.

Examples of Insecticides, Acaricides and Nematicides Which May be Mentioned are:

abamectin, acephate, acrinathrin, alanycarb, aldicarb, alphamethrin, amitraz, avermectin, AZ 60541, azadirachtin, azinphos A, azinphos M, azocyclotin, 4-bromo-2-(4-chlorophenyl)-1-(ethoxymethyl)-5-(trifluoromethyl)-1H-pyrrole-3-carbonitrile, bendiocarb, bensultap, betacyfluthrin, bifenthrin, BPMC, brofenprox, bromophos A, bufencarb, buprofezin, butocarboxin, butylpyridaben, carbaryl, carbofuran, carbophenothion, cartap, chloethocarb, chloretoxyfos, chlorfluazuron, N-[(6-chloro-3-pyridinyl)-methyl]-N′-cyano-N-methyl-ethanimidamide, chlorpyrifos, chlorpyrifos M, cis-resmethrin, clocythrin, clofentezine, cyfluthrin, cyhexatin, cypermethrin, cyromazine, deltamethrin, demeton-M, demeton-S, diafenthiuron, dichlofenthion, dicliphos, dicrotophos, diethiop, diflubenzuron, dimethoate, dimethylvinphos, dioxathion, emamectin, esfenvalerate, ethiofencarb, ethofenprox, fenamiphos, fenazaquin, fenbutatin oxide, fenobucarb, fenothiocarb, fenoxycarb, fenpropathrin, fenpyrad, fenpyroximate, fenvalerate, fipronil, fluazuron, flucycloxuron, flufenoxuron, flufenprox, fonophos, formothion, fubfenprox, hexaflumuron, hexythiazox, imidacloprid, iprobenfos, isazophos, isoprocarb, ivermectin, lambda-cyhalothrin, lufenuron, mevinphos, mesulfenphos, metaldehyde, methacrifos, methamidophos, methidathion, methiocarb, methomyl, metolcarb, milbemectin, monocrotophos, moxidectin, naled, NC 184, nitenpyram, oxamyl, oxydeprofos, permethrin, phosalone, phosmet, pirimicarb, promecarb, propoxur, prothoate, pymetrozine, pyridaphenthion, pyresmethrin, pyridaben, pyrimidifen, pyriproxifen, quinalphos, salithion, sebufos, tebufenozide, tebufenpyrad, tebupirimiphos, teflubenzuron, tefluthrin, temephos, terbam, tetrachlorvinphos, thiacloprid, thiafenox, thiamethoxam, thiodicarb, thiofanox, thiomethon, thuringiensin, tralomethrin, transfluthrin, triarathene, triazuron, trichlorfon, triflumuron, trimethacarb, vamidothion, XMC, xylylcarb, zetamethrin.

Examples of Molluscicides Which May be Mentioned are Metaldehyde and Methiocarb.

Examples of Herbicides Which May be Mentioned are:

Anilides, such as, for example, diflufenican and propanil; arylcarboxylic acids, such as, for example, dichloropicolinic acid, dicamba and picloram; aryloxyalkanoic acids, such as, for example, 2,4-D, 2,4-DB, 2,4-DP, fluroxypyr, MCPA, MCPP and triclopyr; aryloxy-phenoxy-alkanoates, such as, for example, diclofop-methyl, fenoxaprop-ethyl, haloxyfop-methyl and quizalofop-ethyl; azinones, such as, for example, chloridazon and norflurazon; carbamates, such as, for example, chlorpropham, desmedipham, phenmedipham and propham; chloroacetanilides, such as, for example, alachlor, metazachlor and propachlor; dinitroanilines, such as, for example, oryzalin, pendimethalin and trifluralin; diphenyl ethers, such as, for example, acifluorfen, bifenox, fluoroglycofen, fomesafen, halosafen, lactofen and oxyfluorfen; ureas, such as, for example, chlortoluron, diuron, fluometuron, isoproturon, linuron and methabenzthiazuron; hydroxylamines, such as, for example, alloxydim, cycloxydim, sethoxydim and tralkoxydim; imidazolinones, such as, for example, imazethapyr, imazamethabenz, imazapyr and imazaquin; nitriles, such as, for example, bromoxynil, dichlobenil and ioxynil; oxyacetamides, such as, for example, mefenacet; sulfonylureas, such as, for example, amidosulfuron, bensulfuron-methyl, chlorimuron-ethyl, chlorsulfuron, cinosulfuron, metsulfuron-methyl, nicosulfuron, primisulfuron, pyrazosulfuron-ethyl, thifensulfuron-methyl, triasulfuron and tribenuron-methyl; thiolcarbamates, such as, for example, diallate and triallate; triazines, such as, for example, atrazine, cyanazine, simazine, simetryn, terbutryne and terbutylazine; triazinones such as, for example, hexazinon, metamitron and metribuzin; others, such as, for example, aminotriazole, benfuresate, bentazone, clomazone, clopyralid, difenzoquat, dithiopyr, ethoftimesate, fluorochloridone, glufosinate, glyphosate, isoxaben, pyridate, quinchlorac, quinmerac, sulfosate and tridiphane. 4-Amino-N-(1,1-dimethylethyl)-4,5-dihydro-3-(1-methylethyl)-5-oxo-1H-1,2,4-triazole-1-carboxamide and 2-((((4,5-dihydro-4-methyl-5-oxo-3-propoxy-1H-1,2,4-triazol-1-yl)carbonyl)amino)sulfonyl)-methyl benzoate may also be mentioned.

Examples of Plant Growth Regulators Which May be Mentioned are Chlorocholine Chloride and Ethephon.

Dispersants suitable in the case of the formulations of the invention include all customary nonionogenic, anionic, cationic and zwitterionic substances, having the desired surface-active properties, that are normally used in such formulations. These substances include reaction products of fatty acids, fatty acid esters, fatty alcohols, fatty amines, alkylphenols or alkylarylphenols with ethylene oxide and/or propylene oxide, and also their sulphuric esters, phosphoric monoesters and phosphoric diesters, and also reaction products of ethylene oxide with propylene oxide, and also alkylsulphonates, alkyl sulphates, aryl sulphates, tetraalkylammonium halides, trialkylarylammonium halides and alkylamine sulphonates. The dispersants can be used individually or else in a mixture. With preference mention may be made of reaction products of caster oil with ethylene oxide in a molar ratio of from 1:20 to 1:60, reaction products of C₆-C₂₀ alcohols with ethylene oxide in a molar ratio of from 1:5 to 1:50, reaction products of fatty amines with ethylene oxide in a molar ratio of from 1:2 to 1:20, reaction products of 1 mol of phenol with 2 to 3 mol of styrene and from 10 to 50 mol of ethylene oxide, reaction products of C8-C₁₂ alkylphenols with ethylene oxide in a molar ratio of from 1:5 to 1:30, alkylpolyglycosides, C₈-C₁₆ alkylbenzenesulphonic salts, such as calcium salts, monoethanolammonium salts, diethanolammonium salts and triethanolammonium salts, for example.

As examples of nonionic dispersants mention may be made of the products known under the names Pluronic PE 10 100 and Pluronic F 68 (from BASF) and Atlox 4913 (from Uniqema). Also suitable are tristyrylphenyl ethoxylates. As examples of anionic dispersants mention may be made of the product from Bayer AG known under the name Baykanol SL (a condensation product of sulphonated ditolyl ether with formaldehyde), which is available commercially, and phosphated or sulphated tristyrylphenol ethoxylates, in which case specific mention may be made of Soprophor FLK, Soprophor 3D 33 and Soprophor 4D 384 (from Rhodia).

As dispersants mention may also be made by way of example of copolymers of ethylene oxide and propylene oxide, reaction products of tristyrylphenol with ethylene oxide and/or propylene oxide, such as tristyrylphenol ethoxylate containing on average 24 ethylene oxide groups, tristyrylphenol ethoxylate with on average 54 ethylene oxide groups or. tristyrylphenol ethoxylate propoxylate with on average 6 ethylene oxide groups and 8 propylene oxide groups, and also phosphated or sulphated tristyrylphenol ethoxylates, such as phosphated tristyrlphenol ethoxylate with an average of 16 ethylene oxide groups, sulphated tristyrylphenol ethoxylate with an average of 16 ethylene oxide groups or ammonium salt of phosphated tristyrylphenol ethoxylate with on average 16 ethylene oxide groups, and also phospholipids, such as lecithin, optionally as a mixture with gall salts, such as sodium glucocholate, and also liguinsulphonates. Furthermore, substances having wetting agent properties are also suitable. With preference mention may be made of alkylphenol ethoxylates, dialkylsulphosuccinates, such as sodium diisooctylsulphosuccinate, lauryl ether sulphates, and polyoxyethylene-sorbitan fatty acid esters.

Polyvinyl alcohol in the present case refers both to water-soluble polymerization products of vinyl alcohol and to water-soluble, partially hydrolysed polymers of vinyl acetate. Preference is given to polyvinyl alcohol having an average molecular weight of from 10,000 to 200,000.

As an example mention may be made of the product from Clariant known under the tradename Mowiol® 3-83. Preference is also given to partially hydrolysed polyvinyl acetate having an average molecular weight of from 13,000 to 130,000 with an acetate group content of between 1 and 28%. In the case of Mowiol® 3-83 the stated numbers have the following meanings: 3 describes the viscosity of a 4% strength aqueous solution at 20° C. in mPa·s, 83 indicates the degree of hydrolysis in mol %.

Particularly suitable for use in the present case are polyvinyl alcohols obtained by partial hydrolysis of polyvinyl acetate, having a degree of hydrolysis of from 72 to 99 mol % and a viscosity of from 2 to 40 mPa·s, with particular preference between 3 and 18 mPa·s, measured on a 4% strength aqueous solution at 20° C. Both individual examples of these partially hydrolysed polyvinyl acetates and also mixtures are suitable.

Suitable additives which may be present in the formulations of the invention include penetrants, defoamers, low-temperature stabilisers, preservatives, dyes, redispersants, disintegrants, inert fillers, and film formers.

Suitable penetrants in the present context are all substances which are commonly used to enhance the penetration of active agrochemical substances into plants. Preference is given to alkanol alkoxylates of the formula
R—O-(-AO)ₘH (I)
in which
R is straight-chain or branched alkyl having 4 to 20 carbon atoms,
AO is an ethylene oxide radical, a propylene oxide radical, a butylene oxide radical or mixtures of ethylene oxide and propylene oxide radicals, and
m is numbers from 2 to 30.

One particularly preferred group of penetrants are alkanol alkoxylates of the formula
R—O-(-EO—)ₙ—H (Ia)
in which
R is as defined above,
EO is —CH₂—CH₂—O— and
n is numbers from 2 to 20.

Another particularly preferred group of penetrants are alkanol alkoxylates of the formula
R—O(-EO—)ₚ—(—PO—)_{q}—H (Ib)
in which
R is as defined above,
EO is —CH₂—CH₂—O—,
PO is 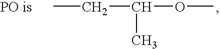
p is numbers from 1 to 10 and
q is numbers from 1 to 10.

Another particularly preferred group of penetrants are alkanol alkoxylates of the formula
R—O(—PO—)ᵣ-(-EO—)ₛ—H (Ic)
in which
R is as defined above,
EO is —CH₂—CH₂—O—, 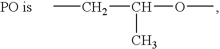
r is numbers from 1 to 10 and
s is numbers from 1 to 10.

Another particularly preferred group of penetrants are alkanol alkoxylates of the formula
CH₃—(CH₂)ₜ—CH₂—O—(—CH₂—CH₂—O—)ᵤ—H (Id)
in which
t is numbers from 8 to 13
and
u is numbers from 6 to 17.

In the formulae indicated above
R is preferably butyl, i-butyl, n-pentyl, i-pentyl, neopentyl, n-hexyl, i-hexyl, n-octyl, i-octyl, 2-ethylhexyl, nonyl, i-nonyl, decyl, n-dodecyl, i-dodecyl, lauryl, myristyl, i-tridecyl, trimethylnonyl, palmityl, stearyl, or eicosyl.

An example of an alkanol alkoxylate of the formula (Ic) is 2-ethylhexyl alkoxylate of the formula 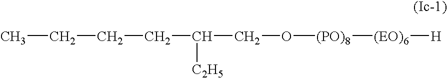
in which
EO is —CH₂—CH₂—O—,
PO is 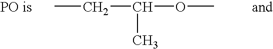
   and the numbers 8 and 6 represent average values.

Particularly preferred alkanol alkoxylates of the formula (Id) are compounds of this formula in which
t is numbers from 9 to 12 and
u is numbers from 7 to 9.

A general definition of the alkanol alkoxylates is given by the formulae above. These substances comprise mixtures of substances of the stated type with different chain lengths. The indices therefore come out at average values, which can in some cases deviate from whole numbers.

By way of example, mention may be made of alkanol alkoxylate of the formula (Id) in which
t is the average value 10.5 and
u is the average value 8.4.

The alkanol alkoxylates of the formulae stated are known or can be prepared by known methods (cf. WO 98-35 553, WO 00-35 278 and EP-A 0 681 865).

Suitable defoamers include all substances which can normally be used for this purpose in agrochemical compositions. Preference is given to silicone oils and magnesium stearate.

Suitable low-temperature stabilisers are all substances which would normally be employed for this purpose in agrochemical compositions. Examples which may be mentioned include urea, glycerol and propylene glycol.

Suitable preservatives include all substances which can normally be used for this purpose in agrochemical compositions of this type. Examples that may be mentioned include Preventol® (from Bayer AG) and Proxel®.

Suitable colorants include all substances which can normally be used for this purpose in agrochemical compositions. Examples that may be mentioned include titanium dioxide, pigment-grade carbon black, zinc oxide and blue pigments and also permanent red FGR.

Suitable redispersants include all substances which can normally be used for this purpose in solid agrochemical compositions. Preference is given to surfactants, swelling agents and sugars. By way of example mention may be made of lactose, urea, polyethylene glycol and tetramethylolpropane.

Suitable disintegrants, as they are known, include substances suitable for accelerating the breakdown of the powder formulations of the invention when combined with water. Preference is given to salts such as sodium chloride and potassium chloride.

Suitable inert fillers include all substances which can normally be used for this purpose in agrochemical compositions. Preference is given to inorganic particles, such as carbonates, silicates and oxides, and also to organic substances, such as urea-formaldehyde condensates. Mention may be made by way of example of kaolin, rutile, silica, highly disperse silica, silica gels, and natural and synthetic silicates, and also talc.

Suitable film formers include water-soluble substances normally used for this purpose in active substance formulations. Preference is given to gelatin, water-soluble starch and water-soluble copolymers of polyvinyl alcohol and polyvinylpyrrolidone.

In the pulverulent active substance formulations of the invention the amount of the individual components can be varied within a relatively wide range. For instance, the concentrations
of solid active substances are generally between 10 and 50% by weight, preferably between 15 and 40% by weight,
of dispersants are generally between 5 and 50% by weight, preferably between 7.5 and 40% by weight,
of polyvinyl alcohol are generally between 10 and 30% by weight, preferably between 15 and 30% by weight, and
of additives are generally between 0 and 50% by weight, preferably between 0 and 40% by weight.

The pulverulent active substance formulations of the invention comprise active substance particles each of which is surrounded by a coat of polyvinyl alcohol, and which are composed of individual particles each surrounded by a matrix of polyvinyl alcohol. The coat-forming polyvinyl alcohol may also comprise other water-soluble, film-forming substances in addition. The particles are in the amorphous state and have an average diameter in the nanometer range. Thus, the average particle diameter of the active substance particles in the polyvinyl alcohol coats (i.e. capsules) is generally between 20 and 1 000 nm, preferably between 50 and 400 nm.

In carrying out the process the procedure of the invention in step (a) is to suspend finely divided, optionally preground active substance and also dispersant and, if desired, additives in water with stirring. This step is generally operated at temperatures of between 10° C. and 30° C., preferably at room temperature.

In the next step (b) of the process of the invention the resulting suspension is heated so that the solid components employed melt and an emulsion is formed in which the melt is distributed in the form of droplets in the aqueous phase. This step is operated at temperatures above the melting point of the respective active substance, generally thus at temperatures between 40° C. and 220° C., preferably between 50° C. and 220° C. Heating preferably takes place at a rate such that the emulsion state is present only for a short time.

The resultant emulsion (i.e. dispersion of melt in droplet form in the water phase) is first of all homogenised in step (c) of the process of the invention, using a jet disperser, so as to give a very fine dispersion. This dispersion is thereafter cooled rapidly to a temperature below the solidification point of the dispersed melt.

Homogenization in a jet disperser, also referred to as melt dispersing, takes place in general at temperatures between 40° C. and 220° C., but in any case above the melting point of the solid components employed. Subsequent cooling is to a temperature below the melting point of the solid components, so that a very fine dispersion of solidified droplets in the aqueous phase is formed. In certain case, however, it is also possible to produce emulsions by cooling to temperatures above the solidification point of the melt.

In the course of melt dispersing it is general procedure to operate at elevated pressure, preferably between 50 bar and 1 600 bar, more preferably between 60 bar and 1 000 bar.

In step (d) of the process of the invention the procedure adopted is to add an aqueous solution of polyvinyl alcohol, alone or in a mixture with other coating materials and also, optionally, with additives to the very fine dispersion prepared, in which the droplets present beforehand have generally solidified, for the purpose of encapsulation, and immediately thereafter to subject the resultant mixture to spray drying.

For the addition of the aqueous solution in step (d) it is general procedure to operate at temperatures between 10° C. and 50° C., preferably between 20° C. and 40° C. For the spray drying step the temperatures can be varied within a relatively wide range. Operation is generally carried out at air entry temperatures of between 100° C. and 200° C., preferably between 120° C. and 180° C., and air exit temperatures of between 50° C. and 100° C., preferably between 60° C. and 90° C.

It is also possible to introduce the emulsion into the spray dryer directly without cooling, i.e. above the melting temperature of the active substance present. In that case cooling only takes place in the spray dryer, in the case for example of cooling to 80° C. Accordingly, the droplets of active substance only solidify after or during the encapsulation to form solid particles in the spray-dried powder.

In one particular variant of the process of the invention it is, however, also possible to use freeze drying to remove the water present. This method is appropriately employed when the active substances are unstable at relatively high temperatures.

Both spray drying and freeze drying are operated so that only a very low level of residual moisture remains in the powder formulation. Drying is generally taken to a point where the residual moisture content lies below 1% by weight.

If aqueous polyvinyl alcohol solution is added as additive as early as in step a) when the process of the invention is being carried out, there is no need to add this capsule-forming coating material in step d).

When conducting the process of the invention, the amounts of the individual constituents are chosen so that in the resultant powder formulation the components are present in the proportions already indicated above.

The Process of the Invention can be Carried Out Either Continuously or Batchwise.

For carrying out the process of the invention it is preferred to use a new apparatus, composed of
a vessel provided with a stirrer and connected via
a heat exchanger,
to which there is connected ajet disperser, from which a pipeline leads
to a cooling circuit provided with a pump, the output line of which is connected to
a metering pump and also, where appropriate, to a mixing vessel, with the pipeline which leads on from the latter
being connected to a spray dryer.

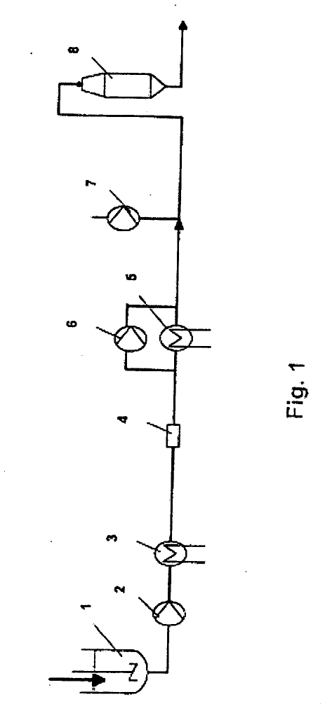

A Diagram of the Apparatus of the Invention is Shown in FIG. 1. In this FIG.

1=vessel provided with stirrer

2=pump suitable for generating pressure

3=heat exchanger

4=jet disperser

5=condenser in cooling circuit

6=pump installed in the cooling circuit

7=metering pump for supplying solution

8=spray dryer

The individual components of the apparatus of the invention are known. Their arrangement in the manner indicated, however, is new.

The heat exchanger 3 is an apparatus which allows rapid heating of the incoming suspension to the desired temperature.

The jet disperser 4 is constructed so that the incoming emulsion is dispersed through a nozzle. The fineness of the dispersion produced is dependent on the homogenising pressure and on the nozzle used. The smaller the nozzle bore, the finer the dispersion obtained. Generally speaking, nozzles are used whose bores are between 0.1 and 1 mm, preferably between 0.2 and 0.7 mm.

The cooling circuit 5 provided with pump 6 is an apparatus suitable for minimizing the cooling time of the incoming emulsion. The emulsion flowing through the condenser 5 is recirculated by means of the pump 6 with approximately ten times the pumped circulation flow rate. This cooling circuit ensures cooling of the admitted emulsion by quenching in the milliseconds range.

The pump 7 is a metering device which is attached to the pipeline; leading away from the cooling circuit. At this point in the apparatus it is also possible to install an additional mixing vessel with stirrer.

The spray dryer 8 is a device of this type which is such that the incoming aqueous solution can have its water removed. The spray dryer may also be replaced by a freeze dryer.

Carrying out the process of the invention using the apparatus of the invention involves in detail, in the first step, suspending one or more solid active substances, where appropriate as a mixture with one or more liquid active substances and also, where appropriate, additives, in a finely divided state in the vessel 1 in a mixture of water and dispersant. The components can be combined in a preground state. Another possibility, however, is to comminute the constituents after they have been mixed, using a rotor/stator disperser, a colloid mill or a bead mill.

The suspension prepared in this way is conveyed by the pump 2 via the heat exchanger 3 into the downstream jet disperser 4. In addition to conveying, the pump also has the function of building up the necessary dispersing pressure. Before entering the jet disperser 4 the dispersion in the heat exchanger 3 is rapidly heated to a temperature above the melting point of the solid phase, so briefly forming an emulsion. This emulsion is then finely homogenised in the jet disperser 4 and immediately after its passage through the jet disperser is cooled in the cooling circuit system 5/6. To minimize the cooling time, the dispersion is passed into the condenser and recirculated using the pump 6 with approximately a tenfold pumped circulation flow. As a result of the cooling loop, quenching ensures that the emulsion is cooled within a period of milliseconds and that the particles of active substance solidify amorphously.

Following passage through the cooling circuit, the metering pump 7 adds an aqueous solution of polyvinyl alcohol and also, where appropriate, further coating materials and/or other additives to the dispersion of amorphous particles. In one particular variant of the process, however, it is also possible for the polyvinyl alcohol solution used to encapsulate the amorphous particles to be added as early as in the cooling circuit 5/6 or even in the batching vessel 1.

Immediately thereafter the solution prestabilized in this way is introduced into the spray dryer 8; here, the water is removed from the dispersion, and the particles of active substance are encapsulated by coating material.

The product in each case is a free-flowing powder. The average size of the particles in the powder depends on the spray drying conditions. Thus, with higher pressure and a greater amount of air, the particles which form are smaller than when using lower pressures and a smaller amount of air.

The formulations of the invention are redispersible powders composed of finely divided active substance particles encapsulated with coating material, the capsules in turn being embedded in coating material.

The powder formulations of the invention are stable even when stored for a relatively long period of time: They can be converted to homogeneous spray liquids by stirring them into water. These spray liquids are used by common methods, i.e., for example, by spraying, pouring or injection. A further option is to granulate the powders or to process them to tablets, pastes or other application forms.

The application rate of the powder formulations of the invention can be varied within a relatively wide range. The rate is guided by the active substances present in each case and by the amount thereof in the formulations.

By means of the powder formulations of the invention it is possible to deliver active substances in a particularly advantageous way. The active substances contained are readily bioavailable and develop a biological activity which is substantially better than that of those conventional formulations in which the active components are present in the crystalline state.

The Invention is Illustrated by the Following Examples.

### PREPARATION EXAMPLES

### Example 1

In a vessel, 3 litres of a suspension consisting of
5 parts by weight of the fungicidal active substance of the formula 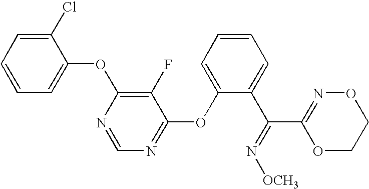
5 parts by weight of emulsifier (phosphoric acid mono-/diester mixture of a tristyrylphenol ethoxylate containing on average 16 ethylene oxide units, tradename Soprophor® 3D33)
0. 1 part by weight of silicone oil defoamer and
89.9 parts by weight of water
   were mixed and, using a bead mill, were ground so that the particle size lies between 1 and 10 μn. The suspension thus prepared was pumped into a heat exchanger, heated to 108° C. and conveyed with a volume flow of 22 litres per hour under a homogenizing pressure of 80 bar into a jet disperser having a nozzle bore of 0.2 mm in diameter. The resultant dispersion, in which the particles have an average size of 0.2 μm, was cooled to a temperature of 30° C. within fractions of seconds, via a cooling circuit, then mixed with 1.2 litres of a 25% strength by weight solution of polyvinyl alcohol (Mowiol® 3-83 from Clariant) in water and immediately thereafter dried by means of a spray dryer with an air exit temperature of <80° C. This gave 550 g of a free-flowing powder having an active substance content of 24.8%, the active substance being in the amorphous state.

### Example 2

In accordance with the method indicated in example 1 a suspension of the composition set out therein was subjected to jet dispersing under the conditions mentioned therein and, after cooling to 30° C., was mixed with 1.543 litres of a 25% strength by weight solution of polyvinyl alcohol (Mowiol® 3-83 from Clariant) in water and with 600 g of primary fatty alcohol ethoxylate (trade name: Dobanol 91-6 from Deutsche Shell Chemie GmbH). Spray drying immediately thereafter gave 1 100 g of a free-flowing powder having an active substance content of 11.86%, the active substance being in the amorphous state.

### Example 3

In a vessel, 3 litres of a suspension consisting of
5 parts by weight of the fungicidal active substance tebuconazole,
5 parts by weight of the emulsifier specified in example 1,
0.1 part by weight of silicone oil defoamer and
89.9 parts by weight of water
   were mixed and, using a bead mill, were ground so that the particle size lies between 1 and 10 μm. The suspension thus prepared was pumped into a heat exchanger, heated to 110° C. and conveyed with a volume flow of 22 litres per hour under a homogenizing pressure of 80 bar into a jet disperser having a nozzle bore of 0.2 mm in diameter. The resultant dispersion, in which the particles have an average size of 0.2 μm, was cooled to a temperature of 30° C. within fractions of seconds by means of a cooling circuit, then mixed with 1.2 litres of a 25% strength by weight solution of polyvinyl alcohol (Mowiol® 3-83 from Clariant) in water and immediately thereafter was dried using a spray dryer with an air exit temperature of <80° C. This gave 550 g of a free-flowing powder having an active substance content of 24.8%, the active substance being in the amorphous state.

### Example 4

In a vessel, 3 litres of a suspension consisting of
2.5 parts by weight of the fungicidal active substance tebuconazole,
2.5 parts by weight of the fungicidal active substance of the formula 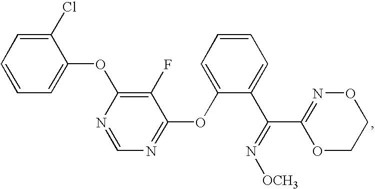
5 parts by weight of the emulsifier specified in example 1,
0.1 part by weight of silicone oil defoamer and
89.9 parts by weight of water
   were mixed and, using a bead mill, were ground so that the particle size lies between 1 and 10 μm. The suspension thus prepared was pumped into a heat exchanger, heated to 110° C. and conveyed with a volume flow of 22 litres per hour under a homogenizing pressure of 80 bar into a jet disperser having a nozzle bore of 0.2 mm in diameter. The resultant dispersion, in which the particles have an average size of 0.2 μm, was cooled to a temperature of 30° C. within fractions of seconds by means of a cooling circuit, then mixed with 1.2 litres of a 25% strength by weight solution of polyvinyl alcohol (Mowiol® 3-83 from Clariant) in water and immediately thereafter was dried using a spray dryer with an air exit temperature of <80° C. This gave 550 g of a free-flowing powder having an active substance content of 24.5%, the active substance being in the amorphous state.

### Comparative Example I

In a vessel, 3 litres of a suspension consisting of
5 parts by weight of the fungicidal active substance of the formula 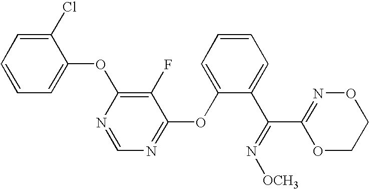
5 parts by weight of the emulsifier specified in example 1,
0.1 part by weight of silicone oil defoamer and
89.9 parts by weight of water
   were mixed and, using a bead mill, were ground so that the particle size lies between 1 and 10 μm. The suspension thus prepared was pumped into a heat exchanger, heated to 108° C. and conveyed with a volume flow of 22 litres per hour under a homogenizing pressure of 80 bar into a jet disperser having a nozzle bore of 0.2 mm in diameter. The resultant dispersion, in which the particles have an average size of 0.2 μm (measured by the photon correlations spectroscopy=PSC method), was cooled to a temperature of 30° C. within fractions of seconds, via a cooling circuit, then dried by means of a spray dryer with a gas exit temperature of 80° C. It was not possible to produce a free-flowing powder. The dried material sticks in the dryer and has a crystalline structure.

### Comparative Example II

In a vessel, 3 litres of a suspension consisting of
5 parts by weight of the fungicidal active substance of the formula 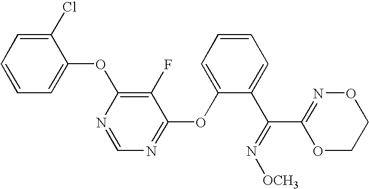
5 parts by weight of the emulsifier specified in example 1,
0.1 part by weight of silicone oil defoamer and
89.9 parts by weight of water
   were mixed and, using a bead mill, were ground so that the particle size lies between 1 and 10 μm. The suspension thus prepared was pumped into a heat exchanger, heated to 108° C. and conveyed with a volume flow of 22 litres per hour under a homogenizing pressure of 80 bar into a jet disperser having a nozzle bore of 0.2 mm in diameter. The resultant dispersion, in which the particles have an average size of 0.2 μm (measured by the photon correlations spectroscopy=PSC method), was cooled to a temperature of 30° C. within fractions of seconds, via a cooling circuit, and then mixed with an amount of 25% strength by weight solution of polyvinyl alcohol (Mowiol® 3-83 from Clariant) in water such that the polyvinyl alcohol fraction in the resultant product was 15% by weight. Subsequent spray drying at an air exit temperature of <80° C. gave 300 g of a powder with a slight degree of agglomeration and with an active substance content of 40%. The crystalline fraction of active substance was 1% and rose during storage of the product.

### Use Example A

Penetration Test

This test measured the penetration of active substance through enzymatically isolated cuticles of apple tree leaves.

The leaves used were cut in free developed state from apple trees of the Golden Delicious variety. The cuticles were isolated as follows:
first of all, leaf discs labelled on the underside with dye and formed by punching were filled by means of vacuum infiltration with a solution (0.2 to 2% strength) buffered to a pH of between 3 and 4,
then sodium azide was added and
the leaf discs thus treated were left to stand until the original leaf structure broke down and the non-cellular cuticles underwent detachment.

Subsequently, only those cuticles from the top leaf sides which were free from stomata and hairs were used. They were washed a number of times in alternation with water and with a buffer solution with a pH of 7. The clean cuticles obtained were, finally, applied to Teflon platelets, smoothed with a gentle air jet and dried.

In the next step, the cuticle membranes obtained in this way were placed in diffusion cells (i.e. transit chambers) made of stainless steel for membrane transport investigations. For these investigations the cuticles were placed centrally using tweezers on the edges of the diffusion cells, which were coated with silicone grease, and were sealed with a ring, which was likewise greased. The arrangement had been chosen so that the morphological outer face of the cuticles was pointing outwards, i.e. to the air, while the original inner face was facing the inside of the diffusion cell. The diffusion cells were filled with water or a mixture of water and solvent.

To determine the penetration, 9 μl of a spray liquor of the composition indicated below were applied to the outer face of each cuticle.

Spray Liquor A (Inventive)

Powder formulation from example 2 in 1 litre of water.

Active Substance Content 1 000 ppm

Spray Liquor B (Known)

Conventional water-dispersible powder of the fungicidal active substance indicated in example 1 in 1 litre of water.

Active substance content 1 000 ppm

CIPAC-C water was used in the spray liquors in each case.

After the spray liquors had been applied the water was evaporated in each case, then each of the chambers was turned round and placed in thermostated troughs, containing in each case a saturated aqueous calcium nitrate tetrahydrate solution below the outer face of the cuticles. The penetration which occurred therefore took place at a relative atmospheric humidity of 56% and a set temperature of 25° C. At regular intervals, samples were taken with a syringe and analyzed by HPLC for the amount of penetrated active substance.

The results of the experiments are evident from the following table. The numbers reported are average values of 8 measurements.

TABLE A

| | Active substance penetration in % after | | |
|---|---|---|---|
| | 24 h | 48 h | 72 h |
| A | 6.4 | 7.1 | 7.7 |
| B | 0.6 | 0.9 | 1.2 |

### Use Example B

Erysiphe Test (Wheat)/Curative

Dispersant: 1 part by weight of the emulsifier specified in example 1

A power formulation according to example 1 is diluted to the desired concentration with water, with addition of dispersant.

To test for curative activity, young plants are dusted with spores of Erysiphe graminis f.sp. tritici. 48 hours after inoculation, the plants are sprayed with the active substance formulation at the application rate indicated.

The plants are placed in a glasshouse at a temperature of approximately 18° C. and a relative atmospheric humidity of approximately 80% in order to promote the development of mildew pustules.

Evaluation takes place 9 days after inoculation. 0% denotes an efficacy which conforms to that of the control, whereas an efficacy of 100% means that no infestation is observed.

Application rates and experimental results are evident from the following table.

TABLE B
Erysiphe Test (wheat)/curative

| Active substance | Application rate of | |
|---|---|---|
| formulation from Example | active substance in g/ha | Efficacy in % |
| Inventive: 1 | 25 | 86 |

1-5. (canceled)

6. A pulverulent active substance formulation comprising:
at least one active substance which is solid at room temperature;
at least one dispersant;
polyvinyl alcohol; and
optionally one or more additives;
the active substance being present in the form of individual particles, said active substance particles being coated with a coat of polyvinyl alcohol, said polyvinyl alcohol coated active substance particles additionally being present in an amorphous state and having a diameter in the nanometer region.

7. A process for producing the pulverulent active substance formulation according to claim 6, comprising:
a) suspending at least one active substance which is solid at room temperature, at least one dispersant, and optionally additives in water, at room temperature;
b) heating the resultant suspension until the solid components it comprises melt to obtain a dispersion comprising said melted solid components in said suspension;
c) homogenizing said first dispersion and then rapidly cooling it to a temperature below the solidification point of said dispersed components to form pulverulent particles of said active substance in said suspension; and
d) thereafter adding an aqueous solution of polyvinyl alcohol, alone or in a mixture with other coating materials and optional additives to the suspension comprising the pulverulent particles of active substance to coat at least a plurality of said particles with a polyvinyl alcohol coating, and immediately thereafter subjecting the resultant suspension of polyvinyl alcohol coated pulverulent particles of active substance to spray drying to obtain the pulverulent active substance formulation according to claim 6.

8. A method of applying an active substance to a target organism and/or its habitat comprising delivering an effective amount of an active substance in a pulverulent active substance formulation according to claim 6, said formulation optionally including one or more extenders and/or surface-active substances, to the target organism and/or its habitat.

9. An apparatus for producing a pulverulent active substance formulation according to claim 6, comprising:
a vessel, said vessel including a stirrer for stirring the contents of said vessel, said vessel having an outlet for the discharge of the contents of said vessel from said vessel;
a first pump in fluid communication with said discharge outlet of said vessel;
a heat exchanger in fluid communication with said first pump;
a jet disperser in fluid communication with said heat exchanger;
a condenser having an inlet and outlet, the inlet of said condenser in fluid communication with said heat exchanger;
a cooling circuit provided about said condenser, said cooling circuit including a second pump and providing a fluid communication loop, said loop in fluid communication from said outlet of said condenser to said inlet of said condenser, said loop providing for the optional recirculation of a fluid from the outlet of said condenser back to the inlet of said condenser through said second pump to cool said fluid;
a metering pump in fluid communication with the outlet of said condenser;
optionally, a mixing vessel in fluid communication with said metering pump; and
a spray dryer in fluid communication with said metering pump or alternatively, in fluid communication with said mixing vessel when said mixing vessel is present.

10. The pulverulent active substance produced by the process of claim 7.

11. A process for producing a pulverulent active substance compris ing the step of producing said pulverulent active substance with the apparatus of claim 9.

12. The pulverulent active substance produced by the process of claim 11.

13. The process of claim 7, further comprising the step of producing said pulverulent active substance with the apparatus of claim 9.

14. The pulverulent active substance produced by the process of claim 13.

15. The method of applying an active substance to a target organism and/or its habitat of claim 8, wherein the pulverulent active substance is produced by the process of claim 7.

16. The method of applying an active substance to a target organism and/or its habitat of claim 8, wherein the pulverulent active substance is produced with the apparatus of claim 9.

17. The method of applying an active substance to a target organism and/or its habitat of claim 8, wherein the pulverulent active substance is produced by the process of claim 7 using the apparatus of claim 9.
